(19) United States
(12) Patent Application Publication
(10) Pub. No.: US 2016/0045614 A1
(43) Pub. Date: Feb. 18, 2016

(54) TARGETED LIPOSOMAL COMPOSITION USING ANTI-HER-2 AFFIBODY MOLECULES AND APPLICATIONS THEREOF IN CANCER TREATMENT

(71) Applicants: Mahmoud Reza Jaafari, Mashhad (IR); Seyyed Mahdi Rezayat, Tehran (IR); Javad Akhtari, Mashhad (IR)

(72) Inventors: Mahmoud Reza Jaafari, Mashhad (IR); Seyyed Mahdi Rezayat, Tehran (IR); Javad Akhtari, Mashhad (IR)

(21) Appl. No.: 14/827,478

(22) Filed: Aug. 17, 2015

Related U.S. Application Data
(60) Provisional application No. 62/038,279, filed on Aug. 17, 2014.

(51) Int. Cl. A61K 47/48; A61K 31/704; A61K 9/127

(52) U.S. Cl. CPC A61K 47/48246; A61K 9/1271; A61K 31/704

(57) ABSTRACT

A liposomal composition for treatment of cancer including a PEGylated liposome loaded by an anthracycline chemotherapeutic agent, wherein the PEGylated liposome is further targeted by an effective number of affibody molecules.

### CROSS REFERENCE TO RELATED APPLICATION

This application claims the benefit of priority from pending U.S. Provisional Patent Application Ser. No. 62/038,279, filed on Aug. 17, 2014, and entitled “Improvement of anticancer efficacy of liposomal doxorubicin by anti-HER-2 affibody mediated targeting for the treatment of breast cancer,” which is incorporated by reference herein in its entirety.

### SPONSORSHIP STATEMENT

This application has been sponsored by the Iranian Nanotechnology Initiative Council, which does not have any rights in this application.

### TECHNICAL FIELD

This disclosure relates to the field of drug delivery, and specifically, liposome-based drug delivery, and more particularly, to the improvement of anticancer efficacy of liposomal doxorubicin by anti-HER-2 affibody-mediated targeting for the treatment of breast cancer.

### BACKGROUND

Traditional chemotherapy is the main treatment for many cancers, but, as is known in the art, its impact is limited due to the toxicity of the agents used in chemotherapy. Since traditional chemotherapy agents are administered systemically and spread all over the body, the toxicity is inevitable. Hence, healthy tissues along with cancerous tissues are equally contacted with powerful chemotherapeutic agents, and irreparable damage could threaten them. As a result, the treatment reduces the quality of a patient's life and medical doctors have to prevent acute and chronic toxicity by prescribing less than optimal amounts of medications.

One solution is drug delivery carriers, within which the cytotoxic drugs can be encapsulated. For example, in nanosized carriers with hydrophilic coatings, the encapsulated drug can be delivered to tumor cells with limited contact with healthy tissues. Liposomes, with biocompatible and biodegradable properties with their specific surface structures are PEGylated with introducing various factors are of those drug carriers.

Doxorubicin (DOX) is an anthracycline chemotherapeutic agent used to treat a variety of cancers. However, doxorubicin is a dose-limited drug because of its cardio-toxicity. In order to address this problem, doxorubicin has been formulated as a PEGylated liposomal composition. The other anthracycline chemotherapeutic agents such as daunorubicin, epirubicin and idarubicin could be used in the same way.

Doxil, which was the first FDA approved nano-drug, is a nanoliposomal form of DOX designed to achieve the goals of the modern chemotherapy. Doxil toxicity profile, compared to traditional therapy with DOX is safer and strongly reduces cardiac toxicity. PEGylation could strongly increase both the stability and the circulation time of doxil. Because of the direct relationship between circulation of liposomes in blood and their accumulation in tumor tissue, the circulation time of the drug is vital for the accumulation of liposomes in tumors. However, the drawback is that, only a very small amount of liposomes enter the cancer cells.

There are some approaches to enhance the selectively of DOX delivery to cancerous cells. One of these approaches is functionalizing liposomes with ligands specific to cancerous membrane receptors. Upon augmentation of liposomes in a tumor's extracellular matrix, the chances of doxorubicin delivery is enhanced due to receptor-mediated endocytosis.

Among ligands for assembling of nanoparticles, affibody molecules hold a better promise compared to antibody molecules and their derivatives. Some characteristics such as enormously smaller hydrodynamic size, resistance to high-temperatures and harsh chemicals, larger affinity for receptors, fast folding kinetics, and that affibody molecules are easier to produce, make them a more suitable option than other antibody molecules for decorating liposomes.

HER2 receptor is a tyrosine kinase receptor, with a molecular weight of 185 kDa, which is recognized as an antigen in breast cancer. HER2/neu expression in 20-30% of breast cancers is considered as too much and is associated with invasive disease and poor prognosis. Several researches have been conducted on the specific delivery of targeted-liposomes containing anti-cancer drugs, using an antibody against HER2. The results have indicated a significant improvement in patients compared to the non-targeted liposomes treatments.

These and many other objects are met in various examples described herein, offering significant advantages over the known prior art and consequent benefits to all mankind.

### SUMMARY

In one general aspect, a liposomal composition for treatment of cancer including a PEGylated liposome loaded by an anthracycline chemotherapeutic agent, wherein the PEGylated liposome is further targeted by an effective number of affibody molecules.

The affibody molecules may specifically bind to at least one extracellular domain of a HER2 receptor.

The affibody molecules may be conjugated with distearoylphosphatidylethanolamine-polyethylene glycol-maleimide (DSPE_EPG_MAL).

The effective number of affibody molecules on the surface of said PEGylated liposome may be less than 50.

The effective number of affibody molecules on the surface of said PEGylated liposome may be less than 40.

The effective number of affibody molecules on the surface of said PEGylated liposome may be between 15 and 25 molecules.

The affibody molecules may be dimer or monomer molecules.

The affibody molecules may be dimer molecules.

The affibody molecules may be head to tail dimer molecules.

The anthracycline chemotherapeutic agent may be selected from the group consisting of doxorubicin, daunorubicin, epirubicin, idarubicin, and their derivatives.

The PEGylated liposome may include cholesterol, phospholipid, and a polyethylene glycol-phospholipid component.

The phospholipid may be selected from the group consisting of hydrogenated soy phosphatidylcholine (HSPC), hydrogenated egg phosphatidylcholine (HEPC), and combinations thereof.

The polyethylene glycol-phospholipid may be DSPE-PEG (2000) [distearoylphosphatidylethanolamine-polyethylene glycol (2000)].

The amount of the phospholipid may range from 40 to 60 mole percent of the total liposomal composition.

The amount of the cholesterol may range from 30 to 40 mole percent of the total liposomal composition.

The amount of said DSPE-EPG may range from 3 to 5 mole percent of the total liposomal composition.

The PEGylated liposome may have an average diameter of about 50 to 150 nanometers.

A ratio of DSPE-EPG-MAL to affibody may be from 8:1 to 12:1.

The affibody molecules may each have a molecular weight of approximately 7000 or approximately 14000 Daltons.

In one general aspect, a method of treating cancer, comprising administering to a human or animal in need thereof a therapeutically effective amount of a liposomal composition including a PEGylated liposome loaded by an anthracycline chemotherapeutic agent, wherein the PEGylated liposome is further targeted by an effective number of affibody molecules.

Other features and aspects will be apparent from the following detailed description, the drawings, and the claims.

### BRIEF DESCRIPTION OF THE DRAWINGS

FIG. 1 illustrates the effect of affibody-conjugation on the hydrodynamic size distribution of liposomes containing Dox (a: Liposomes, b: Affisomes).

FIG. 2 illustrates state of the free peptide band in the SDS-PAGE of affibody-conjugated liposomes and determination of anti-HER2 affibody conjugation to MAL-DSPE-PEG2000.

FIG. 3 illustrates in vitro cellular association of liposomal Dox and affisomes on HER-2 positive cancer cells in comparison to HER-2 negative cells (MDA-MB-231) at 37° C.

FIG. 4 illustrates comparison of the in vitro cytotoxicity of free Dox, liposomal Dox and affisomes with 20 and 40 affibody molecules against human breast adenocarcinoma cells (SKBR3), one hour after administration (a) and after 3 hours after administration (b).

FIG. 5 illustrates the survival cure of different formulation on BALB/c mice. The result of in vivo survival experiments in female BALB/c mice against TUBO in vivo tumor model. BALB/c mice (six in each group) were administrated by intravenous injection of free, liposomal and targeted Dox or PBS after 14 days of tumor inoculation with 5×10⁵ live TUBO cells.

FIG. 6 illustrates tumor growth rate. Tumor growth was measured (three orthogonal diameters) and recorded 3 times per week. Data are presented as mean±standard error mean of 6 mice/group.

FIG. 7 illustrates percentage change due to using different formulation in animal body weight, pursuant to further teachings of this disclosure. Female BALB/c mice inoculated with 10⁵ TUBO tumor cells after 14 days treated with single dose of free and liposomal Dox (15 mg/kg). As before, the data are presented as mean±standard error mean of 6 mice per group.

FIG. 8 illustrates bio-distribution of liposomal DOX and affisomes in organs including kidneys, lungs, heart, spleen, liver, tumor and muscle of BALB/c mice bearing TUBO tumor after administration of a single dose of 15 mg/kg liposomal Dox on day 14 after the tumor inoculation, pursuant to the teachings of this disclosure.

### DETAILED DESCRIPTION

The following detailed descriptions are presented to enable any person skilled in the art to make and use the disclosed subject matter. For purposes of explanation, specific nomenclature is set forth to provide a thorough understanding. However, it will be apparent to one skilled in the art that these specific details are not required to practice the disclosed subject matter. Descriptions of specific applications are provided only as representative examples. Various modifications to the preferred embodiments will be readily apparent to one skilled in the art, and the general principles defined herein may be applied to other embodiments and applications without departing from the scope of this disclosure. The sequences of operations described herein are merely examples, and the sequences of operations are not limited to those set forth herein, but may be changed as will be apparent to one of ordinary skill in the art, with the exception of operations necessarily occurring in a certain order. Also, description of functions and constructions that are well known to one of ordinary skill in the art may be omitted for increased clarity and conciseness. This disclosure is not intended to be limited to the embodiments shown, but is to be accorded the widest possible scope consistent with the principles and features disclosed herein.

In this disclosure, anti-HER2 affibody liposomes (hereinafter “affisomes”) have been developed which possess the tumor cell targeting properties of affibody molecules and the pharmacokinetic and passive targeting properties of PEGylated liposomes.

Development of liposomes modified with affibody molecules is an investigation of the possible effects of coupling molecules on cytotoxicity and cellular uptake. The modified liposomes with affibody (affisome) affected the density of ligand molecules on the surface of liposomes and the way of conjugation.

This disclosure describes a targeted form of liposomal doxorubicin and a safe use of anti-HER2 immunoliposomal anthracyclines to treat HER2-expressing cancers without increasing the cardio toxicity risk as compared to doxorubicin HCl liposome injection (DOXIL), and provides other advantages. It should be understood by those skilled in the art that entrapment of other anthracycline chemotherapeutic agents including, but not limited to, daunorubicon, epirubicin, idarubicin, and their derivatives could be used via the same or similar methods.

The described anti-HER2 targeted liposome (anthracycline-containing immunoliposomes) does not exhibit any more cardio toxicity than doxorubicin HCL liposome injection (DOXIL®), and can be used with the same dosages as with doxorubicin HCL liposome injection, without any increase in cardio toxicity risk or any decrease in its efficacy.

Furthermore, this disclosure demonstrates the efficiency of affisomes with only 20 ligands which effectively target the cells that are expressing HER2, for both in vitro and in vivo conditions.

The Material and Substances

Materials and substances used include: Hydrogenated Soy phosphatidylcholine (HSPC) and Methoxypolyetheleneglycol (Mw 2000)-distearoylphosphatidylethanolamine (mPEG2000-DSPE); Maleimide-PEG2000 distearoylphosphatidylethanolamine (Mal-PEG2000-DSPE); Cholesterol, α-tocopherol, doxorubicin hydrochloride (Dox), phenazinemethosulfate (PMS) and Dowex®; MTS (3-(4,5-dimethylthiazol-2-yl)-5-(3-carboxymethoxyphenyl)-2-(4-sulfophenyl)-2H-tetrazolium, inner salt); Anti-HER2, unconjugated affibody and Anti-HER2, fluorescein conjugated affibody; and Tris (2-carboxyethyl) phosphine hydrochloride (TCEP).

### Preparation of Liposomes

Loaded Liposomes with DOX were prepared by thin film hydration and extrusion methods, respectively. The liposomal formulation was prepared in CHCl₃, by mixing HSPC/cholesterol/PEG-DSPE (in 55, 40, 5, molar ratios respectively). Then, the prepared mixture was dried under a rotary evaporator followed by a high vacuum overnight. The resulting chloroform-free thin films were hydrated with 250 mM NH₄SO₄ and then, extruded through polycarbonate membranes with diameter ranging from 200 nm to 80 nm. Finally, the mixture is loaded by DOX, via an ammonium sulfate gradient method, to achieve Doxil-mimics formulations.

Briefly, 1-5 mg of doxorubicin is added to 5-15 μmol of liposome composition at 65° C. for 60 minutes. Then, it is cooled up to the room temperature, afterwards, it is mixed with Dowex® resin and rotated for 60 minutes to remove the free Dox and, finally it passes through Poly-Prep columns.

Synthesis and Characterization of DSPE-Mal-Affibody

Conjugation of affibody to maleimide-PEG-DSPE was achieved by the following procedure. First, drying of DSPE-PEG-mal from chloroform was carried out under an argon atmosphere. Then, the resulted thin film was hydrated in ddH₂O to prepare micelles. In order to ensure the maximum linkage between the free affibody and the maleimide terminal in micelle, an average maleimide-to-affibody-SH ratios of about 8:1 to 12:1 were used and the resulted mixtures were stirred overnight in a glass tube under an argon atmosphere and light protection. Prior to adding the affibody, TCEP was exploited to reduce any possibility of disulfate linkage among the affibody molecules. This reducing reagent was further removed by dialysis (cut off 100 kDa) after post insertion. Besides, the residual non-reacted maleimide terminals were blocked by cysteine.

To evaluate the post insertion method, affibody molecules are inserted into liposomes containing DilCl8(5) (DiD) by two techniques: post attachment and post insertion which are described in detail as follows:

Post Attachment

A mixture of HSPC:CHO:DSPE-PEG2000-Mal and DiD (55, 40, 5, 0.03 molar ratio) was used to prepare empty liposomes by a film hydration method. The mixture was gently stirred for 8 hours at room temperature. Conjugation of affibody molecules to the liposomal surfaces was achieved by adding 50 to 100 μg of the reduced affibody molecules to about 13 to 15 μmol of lipid to achieve 20 and 40 ligand on the surface of each liposome. Uncoupled peptides were separated from the liposomes by mixture dialysis against dextrose 5% (pH 5.5). The efficiency of the coupling was determined by an SDS-page system. The sizes of the liposomes were determined using a dynamic light scattering method.

Post Insertion

Affibody-PEG2000-DSPE micelles were prepared by using mPEG-DSPE: affibody-PEG-DSPE (in a ratio of 4:1). Then, the micellar dispersion was co-incubated with a preformed plain DiD-liposomes at 60° C. for 60 min. Uncoupled peptides were separated from the liposomes by mixture dialysis methods against the dextrose 5% (pH 5.5). The efficiency of coupling was determined by the SDS-page method, as mentioned before.

Since, the affibody molecules was coupled to the surface of liposomes via a PEGylated lipid, steric constraints would be inevitable (steric constraints were in the liposome bilayers), therefore, dimeric form of affibody ((Z00477)2-Cys) would be an option to overcome the aforementioned steric constraints, since they are head-to-tail dimers through the peptide backbone. The average molecular weight of an affibody molecule is approximately 7 kDa. Obviously, dimeric form will be approximately 14 kDa. The sh-group on the carboxylic end of the first monomer react with maleimide on the PEGylated liposome, and in this way, the second monomer will be exposed on the surface of the liposome. Like other specific interactions in biology, the interaction between the affibody molecules and the extracellular domain of HER2 receptor is very accurate and sensitive. Thus, use of the dimeric form of this ligand decreases constraints and leads to a better interaction and will preserve the functional affibody on the liposomal surface and finally enhances the access to HER2 expressed on target cells.

Characterization of Liposomes

Some liposome characterizations were evaluated including size, zeta potential and leakage stability of liposomes in the presence of 30% serum (RPMI containing 30% FBS). The liposome size and polydispersity index were measured by a Dynamic Light Scattering instrument. Phospholipid content of the prepared composition was measured by a method based on Bartlette phosphate assay to achieve the amount of DOX needed for liposomes loading. In order to determine DOX encapsulation, an appropriate volume of liposomal formulation was dissolved in a solution of acidified isopropyl alcohol (90% isopropyl alcohol; 0.075 M HCl) before and after DOX purification by cationic exchange resins (DOWEX) and measured by spectrofluorimetry (ex: 470 nm/em: 590 nm) through DOX calibration bellow self-quenching concentration. Similarly for the evaluation of the leakage stability of liposomes, a 10-times dilution of the liposomal formulations was prepared through dispersion in RPMI: FCS (70:30 v/v, pH 7.4). Then, aliquots of the media were purified by DOWEX, followed by dissolution in acidified isopropyl alcohol to obtain the amount of DOX retained in the formulation at 0, 1, 2, 4, 6, 12, 24 and 48 hours intervals.

Determination of Affibody Coupling Efficacy

Covalent linkage of affibody to liposomes was determined by polyacrylamide gel electrophoresis in the presence of sodium dodecyl sulfate (SDS-PAGE) under non-reducing conditions. Aliquots of samples including reduced affibody, Doxil® mimic as well as the affibody-conjugated liposomes were mixed with sample buffer containing bromophenol blue as a tracking dye and sodium dodecyl sulfate (SDS) and incubated at 90° C. for 5 min. The samples were then applied onto the polyacrylamide gel, which consists of a running gel (10.22%, w/v, acrylamide) and a stacking gel (4.78%, w/v, acrylamide), at the thickness of 1 mm. The separation was done at the constant voltages of 140 V for 45 min in a vertical slab gel electrophoresis apparatus. The electrophoresis buffer consisted of 25 mM Tris, 192 mM glycine and 0.1% SDS at pH 8.3. The protein bands were developed by a silver staining method.

Evaluation of Anti HER2 Affinity Binding of Affibody Molecules to Cells

Since TUBO cells overexpress rat HER2, and the Affibody used in this study, was anti-human HER2, the binding affinity of SKBR3 and TUBO (HERE2+) was compared to MDA-MB-231(HER2−). MDA-MB-231 cells (HER2−) were used to assess any non-specific interactions. The procedure was as follows: cells were detached from cell culture dishes by using 0.05% trypsin and 0.02% EDTA (GIBCO). Approximately 1000000 cells were incubated with Fluorescein isothiocyanate (FITC)—and then conjugated the affibody molecules in PBS containing 2% FBS and 0.02% sodium azide (PBA) for 1 hour at 4° C. in darkness. Afterward, the cells were washed three times with PBA and re-suspended in an appropriate volume of the buffer and were analyzed by flow cytometry. The collected Data were evaluated with a WinMDI 2.9® program.

Cell Internalization

Three different cell lines, namely, SKBR3, TUBO and MDA-MB-231 were detached by non-enzymatic cell dissociation solution (Millipore, Billerica, Mass.) and 2×10⁵ cells/well were seeded in 24 well plates. The medium was incubated overnight and then was replaced with 1 mL of FCS-free medium containing liposomal preparation at a lipid concentration of 100 nmol of phospholipid/mL and incubated at 37° C. for either 1 or 3 hours. Cells were then washed three times with PBS and detached by 100 μL of trypsin-EDTA solution (Gibco). Then 0.9 mL of an acidified isopropanol was added to each well, and the mixture was incubated overnight at 4° C., to extract the cell-associated Dox. After sedimentation of the cell debris, DOX concentration was measured based on fluorescent excitation at 470 nm and emission at 590 nm, and finally, the percentage of DOX associated with cells was measured.

Cytotoxicity Study:

The anti-proliferative effects of prepared liposomes containing Dox were assessed on SKBR3, TUBO and MDA-MB231 Cells using MTS assay (Promega). For this purpose, the cells were seeded at 5000 cells/well in 96 well plates. After overnight incubation, the medium was replaced with an FCS-free medium containing 1:2 serial dilutions of liposomal Dox or free Dox for scheduled incubation times (1 and 3 hours) at 37° C. The cells were washed gently in PBS and further incubated in the free drugs medium at 37° C. for 72 hours. The viability of cells was determined using a Promega MTS kit. Then, the IC50 of formulations were calculated.

In Vivo Studies: Chemotherapy Study

All animal experiments were conducted in compliance with the Institutional Ethical Committee and Research Advisory Committee of Mashhad University of Medical Sciences guidelines. On day 0, female BALB/c mice, aged 4-6 weeks, were given subcutaneous injections of TUBO tumor cells (5×10⁵ cells per mouse) in the right flank. On day 14, tumor bearing mice with palpable size received medication via a single tail vein injection of either dextrose 5% solution as negative control or doxorubicin at 15 mg/kg encapsulated in liposomes. Mice were weighed and tumor sizes were monitored within 60 days post-injection and the experiment was terminated for those mice that felt so unwell. Kaplain-Meier event free survival probability curve was obtained for each experimental group.

In Vivo Studies: Bio-Distribution Study

Once the size of tumor was grown to nearly 5 mm, the study was begun (almost 14^{th} day after tumor inoculation). For this purpose, mice (4 mice in each group) were injected via the tail vein with 15 mg/kg of either Dox or the Dox loaded liposomal formulations. The Control mice received 200 μL of dextrose 5%. Blood samples were collected via retro orbital bleeding (approx. 0.5 mL) at 6th, 12th and 24^{th} days after dosing. Finally, the groups were sacrificed for tissue collection (the whole tumor, kidneys, spleen, heart, lungs, liver and a portion of muscle) 48 hours after inoculation. The organs were weighted and placed in a 2 mL Polypropylene Microvials (Biospec) containing 1 mL of acidified isopropanol and zirconia beads and homogenized by Mini-Beadbeater-1 (Biospec). After blood coagulation of the blood samples at 4° C., an appropriate amount of sera was diluted in 1 ml of acidified isopropanol. All samples, including the homogenized tissues and sera were stored at 4° C. overnight to extract the drug. The samples were then centrifuged and the supernatant was assayed for Dox concentration spectrofluorimetrically (Ex: 470 nm, Em: 590 nm). The calibration curve was prepared using serial dilutions of Dox in the tissue and sera extracts of the control mice.

### Example 1

### The Properties of Affisome Containing Dox

There are some indications which could be assessed regarding to liposomal formulations. These indications include size of the particulates in the population, the assessment of the affibody molecule linkage to liposomes, and their effect on the release of the drug (here DOX) in simulated conditions.

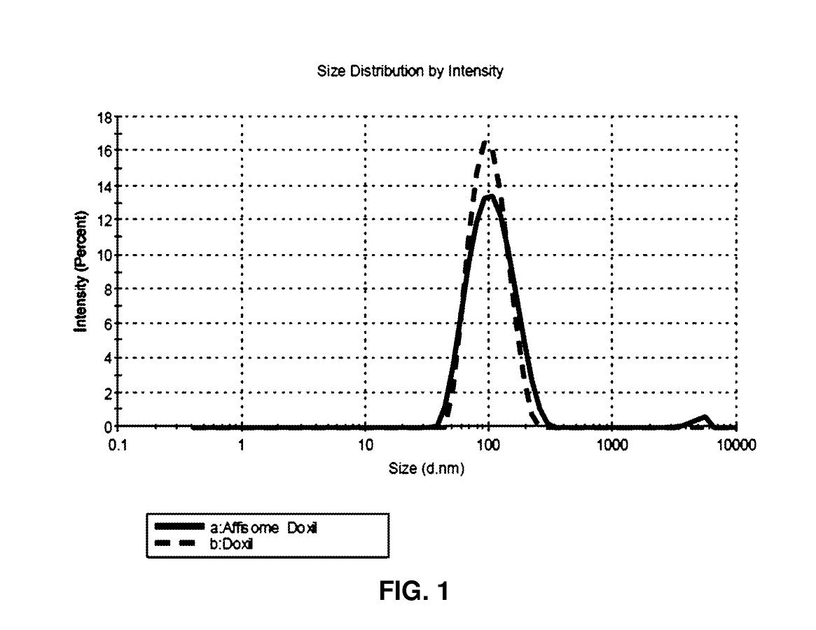

FIG. 1 illustrates the effect of affibody-conjugation on the hydrodynamic size distribution of liposomes containing Dox, the particle size of the liposomes and the affisomes containing Dox with average diameter ranging from 90 to 110 nm and as illustrated in FIG. 1, affibody binding to the surface of the liposomes did not change the mean particle size and Poly Dispersity Index (PDI). In some examples, the PEGylated liposome has an average diameter of about 50 to 150 nm. The physical properties in long-term course of time are set forth and presented in TABLE 1, hereinbelow.

TABLE 1
Physical properties of liposomes containing Dox and its properties
after conjugating to affibody molecules (Affisome).

| | Z-average | | Zeta |
|---|---|---|---|
| Formulation | size (nm)^{a} | Pdi^{a} | potential(mV)^{a} |
| Doxil | 94.05 ± 1.01 | 0.158 ± .005 | −16.6 ± 5.2 |
| Affisome | 98.43 ± 1.32 | 0.185 ± .013 | −17.6 ± 3.8 |

^{a}Mean ± SD (n = 3)

Bivalent affibody molecule was directed against HER2, which possessed a free cysteine residue at the C-terminus, (Z00477)2-Cys for the generation of targeted liposomes. The conjugation of affibody to the liposome surface was based on the chemical reaction between the maleimide of the lipid with the thiol group on the C-terminus of the affibody, as previously described for conjugation of anti-HER2 antibodies to the liposome surface. The used ratio of Affibody:DSPE-MaL-PEG2000 was about 8:1 to 12:1 for reacting to achieve optimal affibody coupling. Affibody molecules were reduced by TCEP before conjugation to break any disulfide linkage.

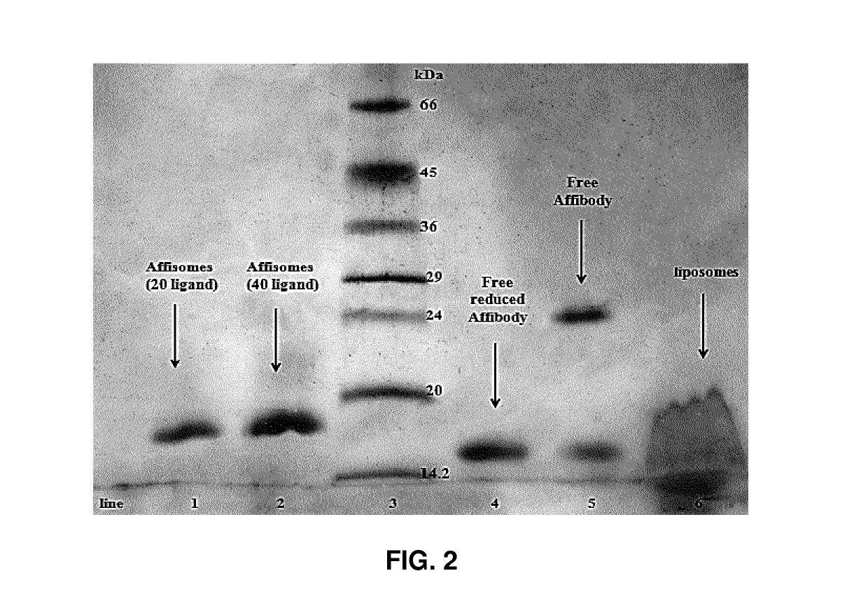

FIG. 2 illustrates the elimination of free peptide band in the SDS-PAGE of affibody-conjugated liposomes (lane 1 and 2), revealed that approximately 100% of the free peptide was consumed. Furthermore, a nearly 3 kDa increase in the molecular weight of the resultant structure confirmed the complete conjugation. On the other hand, liposomes without conjugated affibody (lane 6 in FIG. 2), did not show any bands corresponding to the affibody.

With further reference to FIG. 2, since there is no band in the 14 kDa region at liposome-affibody's region (lane 1 and 2), it can be concluded that linkage efficacy was almost 100%. Consequently, a presumption of 20 and 40 affibody molecules per single liposome was obtained. According to literature review, there are almost 80,000 phospholipid molecules in a single liposome with 100 nm in size. As a result, a 0.0025 to 0.005 mole percent of DSPE-MaL-PEG2000-affibody to liposomes was used in the post insertion process to achieve the final number of 20 to 40 affibody molecules on the surface of each individual liposome.

In all the prepared mixtures, Dox encapsulation efficiencies were above 95%. Leakage stability experiments showed no pronounced differences in Dox release from peptide modified or non-modified liposomes in RPMI; FCS 70, 30 (v/v) at 37° C. within 48 hours incubation. Overall, these findings rule out the probability of aggregation due to presenting peptide segments (affibody molecules) on the surface of liposomes as well as deficiency in Dox containment, regarding the active targeting that peptides could offer.

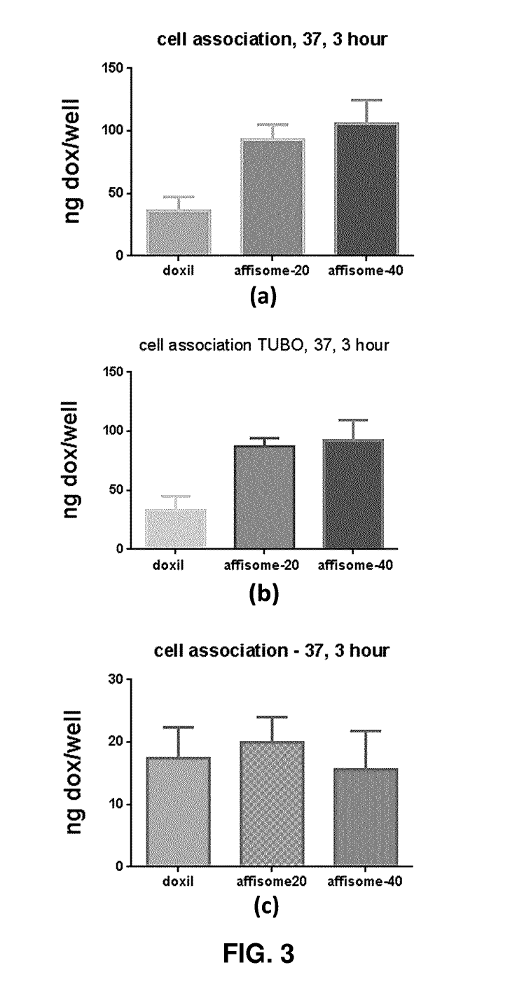

The attachment of the affibody molecule to Mal-PEG2000-DSPE and insertion into PEGylated liposomes increased the specific delivery of Dox to TUBO and SKBR3 against MDA-MB-231 cells by three-folds. The results are illustrated and described in more detail in FIG. 3, wherein (a) illustrates delivery of Dox to SKBR3, (b) delivery of Dox to TUBO, and (c) illustrates delivery of Dox to MDA-MB-231. These results also indicate that, the affinities of anti HER2 affibody molecules are intact and they are being exposed to cells in the liposomal formulations. These results also confirmed that targeting tag is used to direct the PEGylated poly (D, L-lactic acid) nanocomposites to HER positive tumor cells. Alternatively, the targeting efficacy of nanoliposomal Dox, decorated with anti-EGFR affibody to a variety of cancerous cells has been evaluated and no defect has been observed on targeting ability of the affibody molecules. These all, strongly suggest that affibody interaction with the receptors on cells could not be hampered by nanostructures like nanoliposomes and could be used as a way to enhance these drug-nanocarriers' cell internalization.

### Example 2

### The Cytotoxic Effect of Affisome Containing Dox

The cytotoxicity effect of Dox is a matter of its accumulation in cells. The more Dox is delivered to the cells, the more cytotoxic impact could be expected. The incorporation of Dox in liposomal formulation offers the advantage of its accumulation near the tumor cells. However, the slow release of Dox hinders its cytotoxic effect at a concentration necessary to eliminate cancerous cells more efficiently.

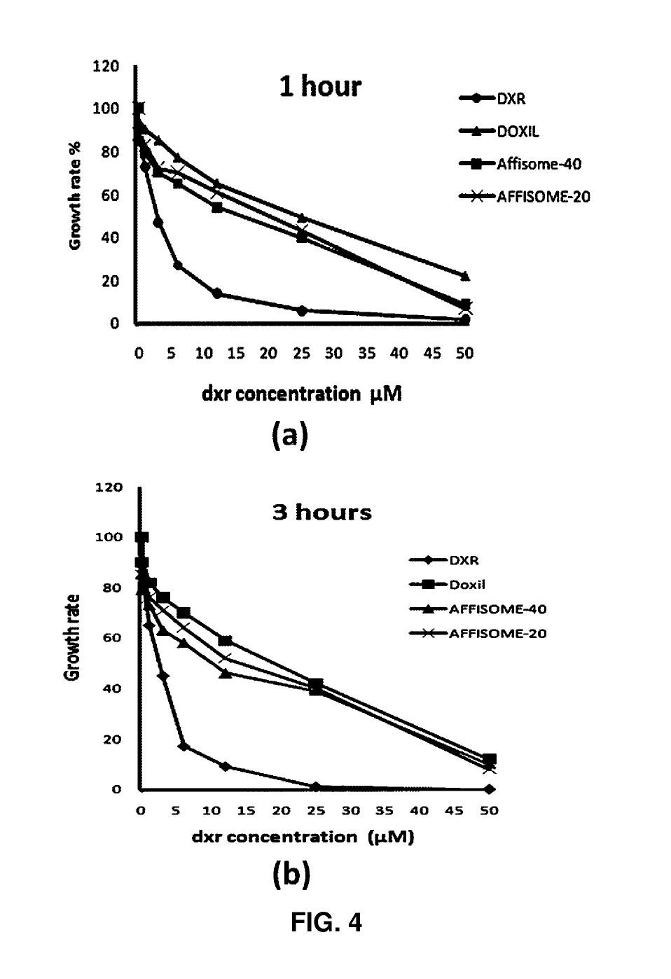

One approach to overcome this issue is the preparation of nanoliposomal Dox conjugated with affibody molecules. The result of cytotoxicity study was in the line of that of “cell association of Dox” portion. The IC50 of free Dox, nanoliposomal Dox and affisomal Dox (with 20 to 40 ligands per liposome), were assessed on HER2+ cell line, SKBR3 and TUBO against HER2− cell line, MDA-MB-231 as illustrated and described in more detail in FIG. 4. Although more uptake and cell killing capability of free Dox was very bolded, there was no distinguishing difference between cytotoxicity of HER2⁺ and HER2⁻ cells. The results for both cells were the same, but with IC50 at far higher concentrations, when nanoliposomal Dox was used. The anti-HER2-affisomal Dox affected specifically only HER2⁺ cell lines, and this effect was ligand-density dependent and time-course dependent.

Understanding the effect of targeting DOX liposomal formulation with affibody molecules as an additional assembly to plain Doxil is of paramount importance.

It is already well-known, that loading DOX in a liposomal formulation with 100 nm in size could dramatically restrict DOX presence in tumor tissues alongside liver and spleen; that means the reduction of its side effects on other vital organs as well as the outcome of therapeutic impact. However, enhancing therapeutic effects without any possible aggregation that the peptide entity may bring about, is a very important factor in targeting nanoliposomal formulation with affibody molecules.

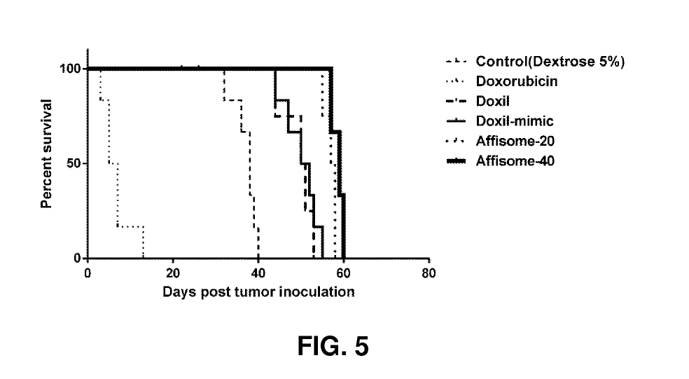

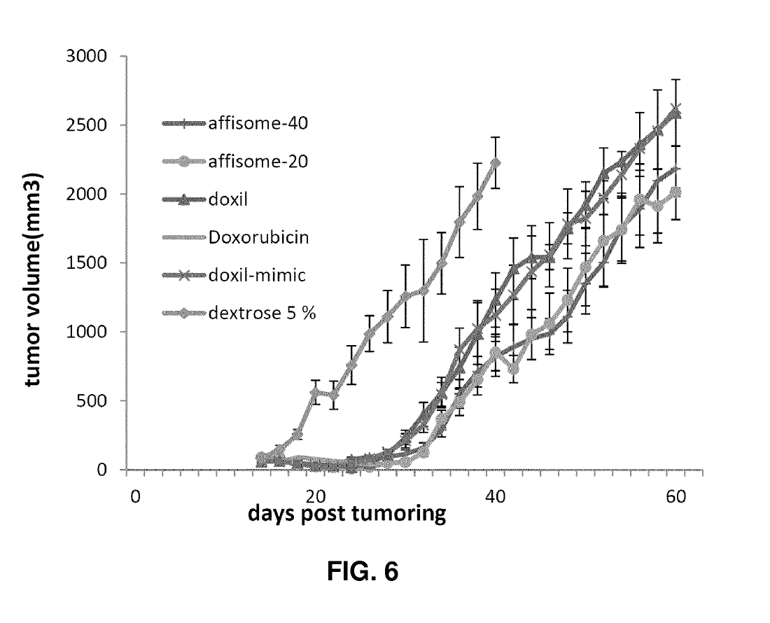

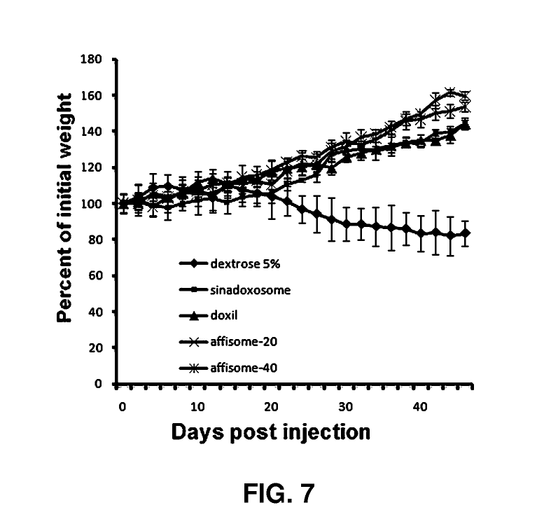

A mouse group treated with the targeted nano-liposomal formulation has the chance to live for a longer time (8 more days) than those who received Doxil, as shown in an event-free survival curve as illustrated in FIG. 5. This result is much more significant compared to control groups (which received sucrose 5%) and those which were treated with Dox. The tumor size in affisome group shrunk earlier and tended to remain constant in size for a longer period of time than Doxil (10 days for the targeted formulation versus 7 for Doxil) as illustrated and described in more detail in FIG. 6. However, the body weight in all groups treated with targeted and non-targeted PEGylated liposomal Dox increased gradually until the last day of experimentation (60 days), which shows that tumor growth was well restricted and Dox toxicity was less. As illustrated and described in more detail in FIG. 7, the body weight profile was plotted against time and presented as percentage of first day body weights. On the other hand, the group which received conventional Dox treatment died in early days possibly because of acute cardiac toxicity.

Although both targeted and non-targeted formulations well stopped the tumor growth, the longer survival for the targeted formulation indicates its better Dox delivery potential. This is in agreement with the in vitro experiments with higher toxicity and more Dox delivery for the targeted liposomal Dox.

Since both targeted and non-targeted liposomal Dox are the same in size, it was already predictable to have similar distribution profiles when they were delivered to circulatory system. PEGylated nano particulate structure like liposomal Dox, accumulation gradually would happen mostly in liver and spleen, the locals of reticulo-endothelial system, and cleared from the circulatory system. In other words, it is easier to contemplate the body, as three compartment, that is, blood, liver, spleen as well as tumor and other organs.

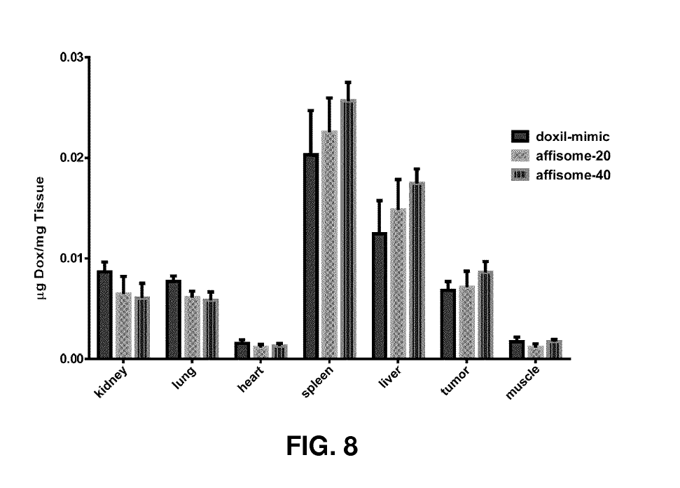

The results on Dox distribution determined that both targeted and non-targeted liposomes cleared virtually from blood within 24 hours post-injection. Targeted liposomal Dox as opposed to plain Doxil, was less recruited to kidney and lungs, but more to liver and tumor, especially for those with 40 ligand as illustrated in more detail in FIG. 8. These results showed the role of reticulo-endothelial cells at scavenging particulate with peptide epitope on surface. However, this effect can be much more tolerable as long as liver biometabolicaly detoxifying pathway alters doxorubicin to doxorubicinol, a non-toxic molecule, than nephrotic and pulmonary system that are regarded as more fragile tissues.

Although the experimental procedure was not able to distinguish the intracellular Dox from those that was contained in liposomes in interstitial fluid, the more detectable Dox in tumors of mice group treated with the targeted liposomal formulation, justifies the cell binding interaction of anti-HER2 affibody to their receptors possibly thrust internalization of liposomal Dox and more restriction to tumor cells.

While the above has provided a description of illustrative embodiments, and while the embodiments have been described in detail, it is not the intention of the Applicant to restrict or in any way limit the scope of the appended claims to such details. Additional advantages and modifications will readily appear to those skilled in the art. Therefore, this application in its broader aspects is not limited to the specific details, representative apparatus and method, and illustrative examples shown and described.

Accordingly, departures may be made from such details without departure from the breadth or scope of the applicant's concept. Furthermore, although the disclosed subject matter has been described in connection with a number of exemplary embodiments and implementations, this application is not so limited but rather covers various modifications and equivalent arrangements, which fall within the purview of the appended claims.

1. A liposomal composition for treatment of cancer comprising:
a PEGylated liposome loaded by an anthracycline chemotherapeutic agent, wherein the PEGylated liposome is targeted by an effective number of affibody molecules.

2. The liposomal composition according to claim 1, wherein the affibody molecules bind to at least one extracellular domain of a HER2 receptor.

3. The liposomal composition according to claim 1, wherein the affibody molecules are conjugated with distearoylphosphatidylethanolamine-polyethylene glycol-maleimide (DSPE_EPG_MAL).

4. The liposomal composition according to claim 1, wherein the effective number of affibody molecules on the surface of said PEGylated liposome is less than 50.

5. The liposomal composition according to claim 1, wherein the effective number of affibody molecules on the surface of said PEGylated liposome is less than 40.

6. The liposomal composition according to claim 1, wherein the effective number of affibody molecules on the surface of said PEGylated liposome is between 15 and 25 molecules.

7. The liposomal composition according to claim 1, wherein the affibody molecules are dimer or monomer molecules.

8. The liposomal composition according to claim 1, wherein the affibody molecules are dimer molecules.

9. The liposomal composition according to claim 1, wherein the affibody molecules are head to tail dimer molecules.

10. The liposomal composition according to claim 1, wherein the anthracycline chemotherapeutic agent is selected from the group consisting of doxorubicin, daunorubicin, epirubicin, idarubicin, and their derivatives.

11. The liposomal composition according to claim 1, wherein the PEGylated liposome comprises cholesterol, phospholipid, and a polyethylene glycol-phospholipid component.

12. The liposomal composition according to claim 11, wherein the phospholipid is selected from the group consisting of hydrogenated soy phosphatidylcholine (HSPC), hydrogenated egg phosphatidylcholine (HEPC), and combinations thereof.

13. The liposomal composition according to claim 11, wherein the polyethylene glycol-phospholipid is DSPE-PEG (2000) [distearoylphosphatidylethanolamine-polyethylene glycol (2000)].

14. The liposomal composition according to claim 11, wherein the amount of the phospholipid ranges from 40 to 60 mole percent of the total liposomal composition.

15. The liposomal composition according to claim 11, wherein the amount of the cholesterol ranges from 30 to 40 mole percent of the total liposomal composition.

16. The liposomal composition according to claim 11, wherein the amount of said DSPE-EPG ranges from 3 to 5 mole percent of the total liposomal composition.

17. The liposomal composition according to claim 1, wherein the PEGylated liposome has an average diameter of about 50 to 150 nanometers.

18. The liposomal composition according to claim 3, wherein a ratio of DSPE-EPG-MAL to affibody is from 8:1 to 12:1.

19. The liposomal composition according to claim 1, wherein the affibody molecules each have a molecular weight of approximately 7000 or approximately 14000 Daltons.

20. A method of treating cancer, comprising administering to a human or animal in need thereof a therapeutically effective amount of a liposomal composition according to claim 1.
